(19) United States
(12) Patent Application Publication
(10) Pub. No.: US 2026/0199420 A1
(43) Pub. Date: Jul. 16, 2026

(54) METHOD FOR PREPARING TEA CAPABLE OF LOWERING BLOOD SUGAR LEVEL

(71) Applicant: POLMEDI CO., LTD., Pocheon-si (KR)

(72) Inventor: Sang-guk LEE, Uijeongbu-si (KR)

(21) Appl. No.: 19/440,985

(22) Filed: Jan. 6, 2026

(30) Foreign Application Priority Data
Jan. 6, 2025 (KR) 10-2025-0001849

(51) Int. Cl. A61K 36/82; A61K 9/00; A61K 33/06; A61K 33/22; A61K 36/06; A61K 36/28; A61K 36/40; A61K 36/42; A61K 36/79; A61K 36/8965; A61P 3/10

(52) U.S. Cl. CPC A61K 36/82; A61K 9/0095; A61K 33/06; A61K 33/22; A61K 36/06; A61K 36/28; A61K 36/40; A61K 36/42; A61K 36/79; A61K 36/8965; A61P 3/10; A61K 2236/37

(57) ABSTRACT

A method for preparing a tea capable of lowering a blood sugar level incudes a first process in which a mineral mixture solution is maintained at room temperature, and then is diluted with to obtain a diluted mineral mixture solution; a second process in which a blood sugar level-lowering mixture is immersed in the diluted mineral mixture solution, and the blood sugar level-lowering mixture coated with the mineral mixture solution is input into a loess jar; a third process of heating the loess jar containing therein the blood sugar level-lowering mixture coated with the mineral mixture solution; a fourth process in which water is input into the jar to obtain an aged product mixed solution and then, the aged product mixed solution is pressurized to extract an extract concentrate; and a fifth process of diluting the extract concentrate with water.

### REFERENCE TO RELATED APPLICATIONS

This application claims the priority benefit of Korean Patent Application No. 10-2025-0001849 filed on Jan. 6, 2025 the entire contents of which are incorporated herein by reference.

### FIELD OF THE INVENTION

The present disclosure relates to a method for preparing a tea of capable of lowering a blood sugar level, and more specifically, to a method for preparing a tea of capable of lowering a blood sugar level, wherein a diabetic patient can simply drink the tea as a health functional food such that the blood sugar level of the diabetic patient can be naturally lowered.

### BACKGROUND OF THE INVENTION

Modern people are undergoing rapid economic development and the accompanying lifestyle and level changes, and their diet is also changed and abundant in various forms, and as a side effect of this, due to excessive consumption of food and biased diet habits, various diseases and diseases such as hyperlipidemia, arteriosclerosis, high blood pressure, and diabetes are rapidly increasing, and interest in health is increasing.

Among these diseases and diseases, diabetes is a condition in which glucose is discharged into the urine due to a high blood sugar level due to a metabolic disorder of carbohydrates, and most of the food consumed is decomposed into glucose by digestive fluid and moved to blood vessels, and enters the cells of our body to be used as a growth and energy source. Insulin must be present when glucose enters the cell, and insulin is produced in the pancreas. The pancreas automatically produces a moderate amount of insulin to move glucose from the blood to the cells when eating food, and diabetic patients have various abnormal symptoms because the pancreas hardly produces insulin or the cells do not respond to insulin, so glucose cannot enter the cells, remain in the blood, and are excreted into the urine, so there is a lot of glucose in the blood, but the body cannot use it.

These diabetes mellitus are classified into several categories according to their type, and first, type 1 diabetes, also called insulin-dependent or childhood diabetes, is diabetes caused by autoimmune diseases. In other words, our body's immune system, which is supposed to fight infection from the outside, destroys beta cells that secrete insulin from the pancreas, so insulin is not secreted at all or secretion is reduced, and the cause of the immune system's attack on beta cells is not yet known.

Type 1 diabetes accounts for less than 10% of all diabetes mellitus and occurs mainly in children and young adults, but can occur at any age, and the symptoms include thirst, increased urination, continued hunger, weight loss, decreased vision, extreme fatigue, and if not treated, life-threatening coma. Second, there is type 2 diabetes, also called insulin-independent diabetes. It accounts for more than 90% of all diabetes, mainly after the age of 40, and patients are usually obese. In the case of type 2 diabetes, the pancreas secretes insulin, but the blood sugar increases due to the inability to effectively utilize the insulin secreted by the body. It can be treated with oral hypoglycemic agents and insulin. Symptoms appear slowly and are not as pronounced as symptoms of type 1 diabetes. Symptoms include fatigue, frequent urination, thirst, weight loss, decreased vision, frequent infection, and wounds do not heal well. Third, there is gestational diabetes, which refers to diabetes that occurs newly during pregnancy or is diagnosed for the first time during pregnancy, and it generally disappears when the pregnancy is terminated due to childbirth, but there is a high risk of developing type 2 diabetes later. In addition, it refers to diabetes that is classified as diabetes caused by other factors and appears due to decreased insulin activity due to certain drugs, hormones, and genetic diseases, and includes diabetes caused by pancreatitis or pancreatic cancer.

On the other hand, the ultimate purpose of diabetes treatment can be said to be to prevent complications while maintaining blood sugar level normally, and the health care methods of diabetic patients can be broadly divided into three types: diet, exercise therapy, and drug therapy.

In order to prevent arteriosclerosis by reducing the resistance to insulin and reducing blood sugar, and facilitating lipid metabolism, health can be managed through therapy according to the patient's condition, and in case of drug therapy, there are largely oral hypoglycemic drugs and insulin therapy, dose, drug should be changed while regular blood sugar tests are performed, and side effects and complications should be carefully examined. However, for the treatment of diabetes at present, there are inconveniences and risks of having to be inoculated with insulin injection, which is the only hormone that lowers blood sugar in the body, and in the case of drugs being used, the continuous medicinal effects are insufficient and various side effects may occur in the body, and in particular, many therapeutic agents have problems causing renal toxicity and hepatotoxicity. Therefore, various attempts have been made to develop a new natural diabetes treatment agent having excellent pharmacological effects while reducing such discomfort and side effects.

On the other hand, due to changes in living culture, there is a tendency to expect various side effects through the intake of convenient foods as possible, and various developments are being made for tea products with enhanced convenience and functionality in accordance with this. Aging, which also refers to a process of aging, is a process in which molecules mixed with each other in the natural state react to have a new structure, and those that are not evenly mixed with each other are decomposed into small molecular structures while visiting their place. Aging in the natural state takes a very long time, and the time required determines the flavor of food or the efficacy of a substance component.

### PRIOR ART LITERATURE

### Patent Document

(Patent Document 1) Korean Patent No. 10-1200344
(Patent Document 2) Korean Patent No. 10-1331238
(Patent Document 3) Korean Patent No. 10-1369507
(Patent Document 4) Korean Patent No. 10-2622081

### SUMMARY OF THE INVENTION

The present disclosure is devised to solve the above problems. A purpose of the present disclosure is to provide a method for preparing a tea of capable of lowering a blood sugar level, in which activation of the aging and the maximum extraction of effective ingredients of the mixture of materials that lower the blood sugar level may be achieved via ionic action of minerals and heating, thereby effectively lowering the blood sugar level of diabetic patients simply drinking the tea as the health functional food, and helping prevent diabetes and complications.

In order to achieve the purpose, a method for preparing a tea capable of lowering a blood sugar level incudes a first process in which a mineral mixture solution is maintained at room temperature, and then is diluted with to obtain a diluted mineral mixture solution; a second process in which a blood sugar level-lowering mixture is immersed in the diluted mineral mixture solution, and the blood sugar level-lowering mixture coated with the mineral mixture solution is input into a loess jar; a third process of heating the loess jar containing therein the blood sugar level-lowering mixture coated with the mineral mixture solution; a fourth process in which water is input into the jar to obtain an aged product mixed solution and then, the aged product mixed solution is pressurized to extract an extract concentrate; and a fifth process of diluting the extract concentrate with water.

In order to achieve the purpose, the present disclosure provides a method for preparing a tea capable of lowering a blood sugar level, the method comprising: a first process in which 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain a mineral mixture solution, and then, the mineral mixture solution is maintained at room temperature for 24 to 36 hours, and then is diluted with water at a ratio of 10 to 15 g of the mineral mixture solution per 1 kg of the water to obtain a diluted mineral mixture solution; a second process in which 30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture, and then, the blood sugar level-lowering mixture is immersed in the diluted mineral mixture solution for 20 to 30 minutes, and is removed from the diluted mineral mixture solution to obtain the blood sugar level-lowering mixture coated with the mineral mixture solution, and then the blood sugar level-lowering mixture coated with the mineral mixture solution is input into a loess jar; a third process of heating the loess jar containing therein the blood sugar level-lowering mixture coated with the mineral mixture solution to 30 to 50° C. for 12 to 24 hours to obtain an aged product; and a fourth process in which water is input into the jar at the same weight amount as a weight amount of the aged product to obtain an aged product mixed solution and then, the aged product mixed solution is pressurized to extract an extract concentrate; and a fifth process of diluting the extract concentrate with water at a ratio of 1:100.

According to the method for producing the tea capable of lowering the blood sugar level of the present disclosure, the activation of the aging and the maximum extraction of effective ingredients of the mixture of materials that lower the blood sugar level may be achieved via ionic action of minerals and heating, thereby effectively lowering the blood sugar level of diabetic patients simply drinking the tea as the health functional food, and helping prevent diabetes and complications.

In the method for preparing the tea capable of lowering the blood sugar level according to an embodiment of the present disclosure, the aging of the materials including the green tea may be activated and continuously maintained under the ionic action of mineral components such as tourmaline added into water, and the ionic action becomes more active via the external heating of the jar into which the materials are put, so that the intrinsic effective ingredients of the materials may be maximally extracted and be well absorbed into the body of a diabetic patient, thereby alleviating or eliminating symptoms caused by insulin deficiency. Thus, the intrinsic effective ingredients of the blood sugar level lowering mixture can be absorbed well into the body of a diabetic patient so as to relieve or eliminate symptoms caused by a shortage of insulin. Furthermore, the tea of the present disclosure can be easily drunk as the health functional food, thereby reducing the resistance to general herbal medicines and safely and naturally reducing the blood sugar level without side effects by replacing chemically synthesized diabetes therapeutic agents, thereby helping to treat diabetes and prevent complications.

### BRIEF DESCRIPTION OF THE DRAWINGS

FIG. 1 is a flowchart illustrating a process of preparing a tea capable of lowering a blood sugar level according to an embodiment of the present disclosure.

FIGS. 2A and 2B are captured images of heating of a loess jar into which materials including green tea are added during a process of preparing the tea capable of lowering the blood sugar level of FIG. 1.

FIG. 3 is a captured image showing a state of the materials including the green tea after the heating in FIGS. 2A and 2B.

FIG. 4 is a diagram showing a relationship between cell viability based on a concentration of the tea capable of lowering a blood sugar level as prepared through the process of FIG. 1 in an experiment of transporting glucose into muscle cells of a mouse using the tea.

FIG. 5 is a diagram showing glucose absorption in the experiment of transporting glucose into the muscle cells of a mouse using insulin and the tea capable of lowering the blood sugar level as prepared through the process of FIG. 1.

FIGS. 6A to 6C are diagrams illustrating an example of change in the blood glucose level before and after drinking the tea capable of lowering a blood sugar level as produced through the process of FIG. 1.

### DETAILED DESCRIPTION OF THE INVENTION

Hereinafter, embodiments of the present disclosure will be described in detail with reference to the accompanying drawings. The present disclosure may be subjected to various changes and may have various forms. Thus, particular embodiments will be illustrated in the drawings and will be described in detail herein. However, this is not intended to limit the present disclosure to a specific disclosed form. It should be understood that the present disclosure includes all modifications, equivalents, and replacements included in the spirit and technical scope of the present disclosure. While describing the drawings, similar reference numerals are used for similar components. In the accompanying drawings, the dimensions of the structures are shown in an enlarged manner for clarity of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “including”, “include”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

In interpreting a numerical value, the value is interpreted as including an error range unless there is no separate explicit description thereof.

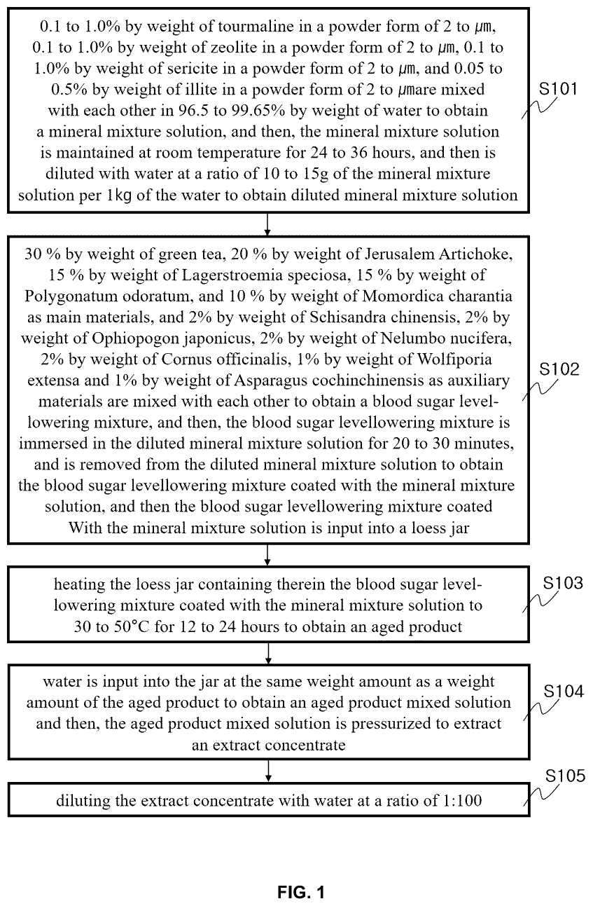

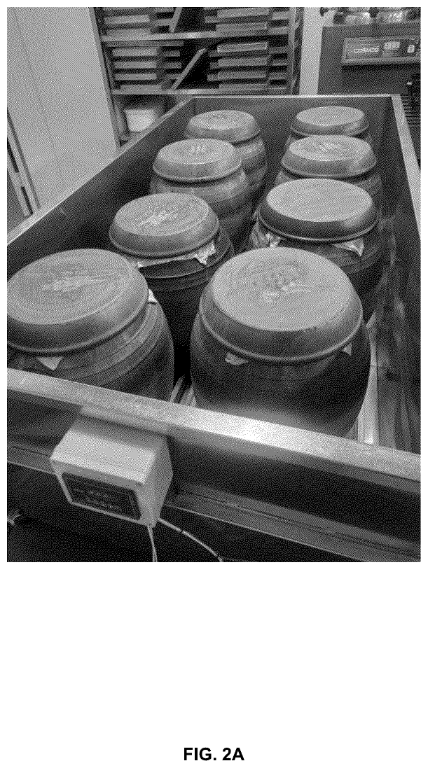

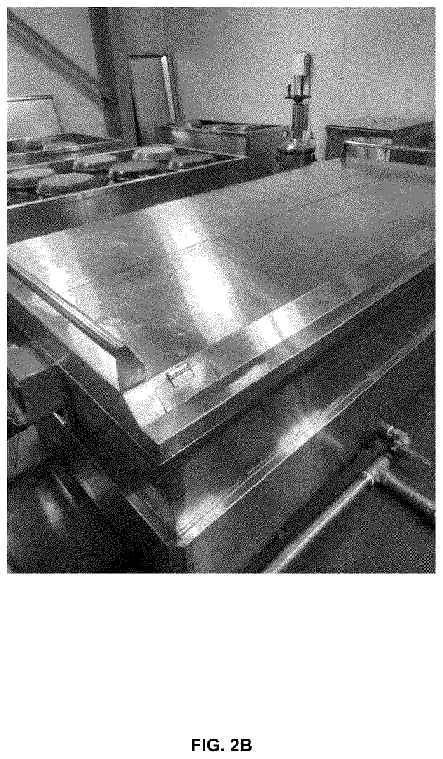

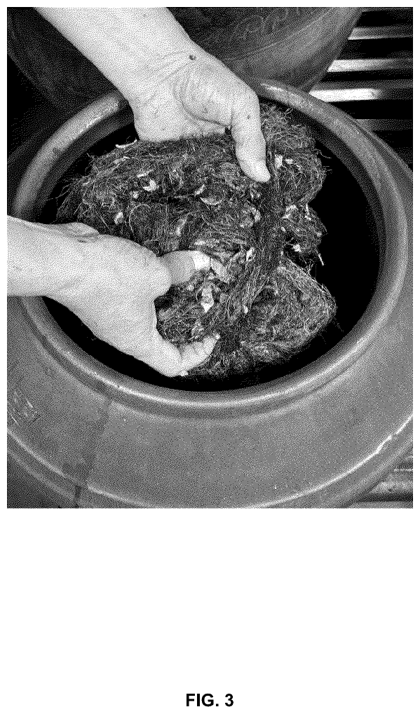

FIG. 1 is a flowchart illustrating a process of preparing a tea capable of lowering a blood sugar level according to an embodiment of the present disclosure. FIGS. 2A and 2B are captured images of heating of a loess jar into which materials including green tea are added during a process of preparing the tea capable of lowering the blood sugar level of FIG. 1. FIG. 3 is a captured image showing a state of the materials including the green tea after the heating in FIGS. 2A and 2B.

In the method for preparing the tea capable of lowering the blood sugar level according to an embodiment of the present disclosure, the aging of the materials including the green tea may be activated and continuously maintained under the ionic action of mineral components such as tourmaline added into water, and the ionic action becomes more active via the external heating of the jar into which the materials are put, so that the intrinsic effective ingredients of the materials may be maximally extracted and be well absorbed into the body of a diabetic patient, thereby alleviating or eliminating symptoms caused by insulin deficiency.

Specifically, as shown in FIG. 1, the method for preparing the tea capable of lowering a blood sugar level according to an embodiment of the present disclosure includes a first process S101 in which 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain a mineral mixture solution, and then, the mineral mixture solution is maintained at room temperature for 24 to 36 hours, and then is diluted with water at a ratio of 10 to 15 g of the mineral mixture solution per 1 kg of the water to obtain a diluted mineral mixture solution; a second process S102 in which 30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture, and then, the blood sugar level-lowering mixture is immersed in the diluted mineral mixture solution for 20 to 30 minutes, and is removed from the diluted mineral mixture solution to obtain the blood sugar level-lowering mixture coated with the mineral mixture solution, and then the blood sugar level-lowering mixture coated with the mineral mixture solution is input into a loess jar; a third process S103 of heating the loess jar containing therein the blood sugar level-lowering mixture coated with the mineral mixture solution to 30 to 50° C. for 12 to 24 hours to obtain an aged product; and a fourth process S104 in which water is input into the jar at the same weight amount as a weight amount of the aged product to obtain an aged product mixed solution and then, the aged product mixed solution is pressurized to extract an extract concentrate; and a fifth process S105 of diluting the extract concentrate with water at a ratio of 1:100. The method can produce the tea as a health functional food which the person having diabetes can easily drink, thereby reducing the resistance to general herbal drugs and safely and replacing chemically synthesized diabetes treatment agents, and naturally reducing the blood sugar level without side effects, thereby helping to treat diabetes and prevent complications.

First, in the first process S101, 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain a mineral mixture solution, and then, the mineral mixture solution is maintained at room temperature for 24 to 36 hours, and then is diluted with water at a ratio of 10 to 15 g of the mineral mixture solution per 1 kg of the water to obtain a diluted mineral mixture solution.

In an embodiment of the present disclosure, tourmaline contained in the mineral mixture solution is a mineral in which a crystal itself has a characteristic of constantly generating electricity, and Mg, Fe, B, Si, and Ca having a (+) pole and a (−) pole at both ends of the crystal, respectively, no matter how small the crystal is are main components, and anions generated by irregular shaking of natural atoms in a complex structure are variously used.

The tormaline continuously transfers the generated ions to zeolite, sericite, and illite to promote, strengthen, and maintain ionization between mineral ions. 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain 100 to form the mineral mixture solution of 100% by weight. The content of the tourmaline is in in a range of 0.1 to 1.0% by weight based on the total 100% of the mineral mixture solution.

The zeolite contained in the mineral in an embodiment of the present disclosure is an inorganic material that contains exchangeable cations in a crystal structure and is freely exchanged with other cations, and is generally used to remove and purify various harmful substances among materials such as green tea, remove and adsorb various heavy metals, and deodorize and purify them.

The zeolite maintains a strongly ionized state through ion exchange with tourmaline, sericite, and illite mineral ions. The content of the zeolite is in in a range of 0.1 to 1.0% by weight based on the total 100% of the mineral mixture solution.

In an embodiment of the present disclosure, the sericite contained in the mineral mixture solution include SiO2, Al2O3, Fe2O3, CaO, MgO, K2O, and Na2O as main components and is rich in dozens of essential minerals, trace elements, and the like, has excellent adsorption power by emitting more than 90% of far-infrared rays having a wavelength of 5 to 20 μm at room temperature and strong negative ions, has antibacterial and antifungal effects, and has odor removal effects, and prevents contamination of materials such as green tea by causing precipitation through electrical neutralization coagulation with particles, and activates materials by neutralizing and detoxifying various pollutants.

The sericite increases the generation of tourmaline, zeolite, and illite mineral ions to maintain a strongly ionized state. The content of sericite is in in a range of 0.1 to 1.0% by weight based on the total 100% of the mineral mixture solution.

In an embodiment of the present disclosure, illite contained in the mineral mixture solution is a common mineral in sedimentary rocks or hydrothermal metamorphic rocks, and has a mineral structure in a clay structure, chemical components are composed of SiO₂, Al₂O₃, and K₂O, and is formed at a relatively high temperature compared to other clay minerals, and K+ is present between layers, and thus it is expected that the utilization thereof is increased from an environmental aspect.

The illite maintains a strongly ionized state in a balance with tourmaline, zeolite, and sericite mineral ions. The content of illite is in a range of 0.05 to 0.5% by weight based on the total 100% of the mineral mixture solution.

In addition, the aging of the blood sugar level lowering materials including the green tea may be activated and continuously maintained under the ionic action of the mineral components such as tourmaline added into water. In this regard, this effect may be maximized when the content of the tourmaline is in in a range of 0.1 to 1.0% by weight based on the total 100% of the mineral mixture solution, the content of the zeolite is in in a range of 0.1 to 1.0% by weight based on the total 100% of the mineral mixture solution, the content of sericite is in in a range of 0.1 to 1.0% by weight based on the total 100% of the mineral mixture solution and the content of illite is in a range of 0.05 to 0.5% by weight based on the total 100% of the mineral mixture solution.

In addition, 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain the mineral mixture solution, and then, the mineral mixture solution is maintained at room temperature for 24 to 36 hours, such that the ions generated from tourmaline increase ion generation of respective mineral ions of zeolite, sericite, and illite, and ion exchange is continuously performed while maintaining ion balance, thereby promoting and strengthening mineral ionization, and forming and maintaining the aging activation base of the materials such as green tea while maintaining a stronger ionization state.

Next, in the second process S102, 30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture, and then, the blood sugar level-lowering mixture is immersed in the diluted mineral mixture solution for 20 to 30 minutes, and is removed from the diluted mineral mixture solution to obtain the blood sugar level-lowering mixture coated with the mineral mixture solution, and then the blood sugar level-lowering mixture coated with the mineral mixture solution is input into a loess jar.

The blood sugar level-lowering mixture is immersed in the diluted mineral mixture solution for 20 to 30 minutes, thereby activating the aging of the blood sugar level-lowering materials under the ionic action of the mineral mixture solution.

In this regard, 30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture.

First, the green tea contains various bioactive substances, which may have a positive effect on blood sugar control and insulin sensitivity improvement.

Specifically, green tea contains catechin, a potent antioxidant such as epigallocatechin gallate (EGCG). EGCG helps lower blood sugar level and improve insulin sensitivity. In addition, the antioxidant of green tea reduces oxidative stress, which is advantageous in preventing complications related to diabetes. Antioxidant action contributes to preventing cell damage and improving overall health. In addition, green tea may help reduce postprandial blood glucose rise by inhibiting the absorption of sugars in the small intestine. This contributes to preventing blood glucose spikes and maintaining stable blood glucose levels. In addition, the anti-inflammatory properties of green tea help to reduce the inflammatory response associated with diabetes. Inflammation is associated with insulin resistance, so it is important to reduce it. In addition, green tea can promote weight loss and body fat loss. Weight management is an important factor in improving insulin sensitivity and preventing type 2 diabetes.

Next, Lagerstroemia speciosa called as Banaba has physiological effects that are good for diabetes, and specifically, Lagerstroemia speciosa leaves are rich in a compound called corosolsan. Corosol acid is known to be effective in lowering blood sugar, and it helps control blood sugar by improving insulin sensitivity. In addition, the vanaba extract helps reduce the rise in blood sugar after meals by inhibiting glucose absorption in the intestine. This is advantageous for diabetes management by preventing blood sugar spikes. In addition, the Lagerstroemia speciosa extract is rich in antioxidants to reduce oxidative stress, which helps prevent complications related to diabetes. In addition, the Lagerstroemia speciosa extract promotes fat decomposition in fat cells, thereby helping to reduce weight and body fat. This has a positive effect on improving insulin resistance and preventing type 2 diabetes. In addition, the vanaba extract shows insulin-like effects and can effectively lower blood sugar even if it does not promote insulin secretion.

Next, Momordica charantia called as the bitter melon has good physiological effects for diabetes, and specifically, Momordica charantia contains a compound called Charantin. Charantine has the effect of lowering blood sugar and helps control blood sugar through an action similar to insulin. Momordica charantia contains an insulin-like protein called polypeptide-P. It is effective in lowering blood sugar and can be used as an insulin substitute. In addition, in Momordica charantia, Vicarantin and polypeptide components help regulate glucose metabolism and stabilize blood sugar level. In addition, Momordica charantia is rich in antioxidants to reduce oxidative stress, which helps prevent complications related to diabetes. Antioxidant action prevents cell damage and promotes overall health. In addition, Momordica charantia helps reduce the rise in blood sugar after meals by inhibiting the absorption of glucose in the intestine. This contributes to preventing blood glucose spikes and maintaining stable blood glucose levels. In addition, Momordica charantia can improve blood sugar control by promoting insulin secretion. This may be particularly advantageous for people with insulin resistance.

Next, Jerusalem Artichoke have physiological effects that are good for diabetes, and specifically, Jerusalem Artichoke contain a large amount of dietary fiber called Inulin. Inulin is not digested in the small intestine but moves to the large intestine and acts as a prebiotic. This helps keep blood sugar stable without raising it rapidly. In addition, inulin has a low blood sugar index, which suppresses the rise in blood sugar and helps to maintain a stable blood sugar level after meals. This is particularly advantageous for diabetic patients. In addition, inulin and other fibers from Jerusalem Artichoke can improve insulin sensitivity. This contributes to reducing insulin resistance and helping blood sugar control. In addition, Jerusalem Artichoke improve intestinal health by promoting the growth of beneficial bacteria in the intestine. Improved intestinal health can also have a positive effect on overall metabolic health. In addition, the dietary fiber of Jerusalem Artichoke increases satiety, preventing overeating and helping weight management. Weight management is one of the important factors in diabetes management. In addition, since Jerusalem Artichoke contain antioxidants, they reduce oxidative stress, which helps prevent diabetes-related complications.

Next, Polygonatum odoratum is used as a traditional herbal medicine and may be helpful in diabetes management, and specifically, Polygonatum odoratum has an effect of promoting insulin secretion. This helps control blood sugar. Studies have shown that Polygonatum odoratum extract may have a positive effect on increasing insulin secretion and improving insulin sensitivity. In addition, the roots of Solomon's seal contain ingredients that help lower blood sugar level. This is advantageous for diabetics to manage blood sugar. In addition, Solomon's seal roots are rich in antioxidants, helping to reduce oxidative stress. Antioxidant action can have a positive effect on preventing cell damage and preventing diabetes-related complications. In addition, Solomon's seal root has an anti-inflammatory effect to reduce inflammation, which helps to relieve the inflammatory response related to diabetes. In addition, L. scutellata roots can contribute to improving weight management and metabolic health by promoting metabolism and optimizing energy use. This is an important factor in diabetes management.

In addition, as an auxiliary ingredient, Schisandra chinensis is named as five-flavor berry because it has five flavors, and it protects the essence, relieves thirst, and helps recover from fatigue and improve physical strength. It is also known to help control blood sugar. Big blue lilyturf cools the lungs and stomach, and replenishes the essence to relieve thirst. It is useful for relieving thirst and thirst symptoms, so it helps relieve thirst caused by diabetes. Lotus jade protects the stomach, helps digestion, and replenishes the essence to enrich the body. It is known that it can also help control blood sugar. Cornus officinalis strengthens the liver and kidneys, replenishes the essence, and has the effect of improving physical strength. In particular, it is known as a drug that can help recover from fatigue and control blood sugar. Wolfiporia extensa has a diuretic effect, which helps the body to metabolize water, promotes digestion, and has a soothing effect. It is beneficial in quenching the thirst caused by diabetes. The astronomy room replenishes the essence, protects the lungs and kidneys, and has an excellent effect on quenching thirst. In addition, it has the effect of calming the body and lowering fever, which can be helpful for diabetic patients.

30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture, thereby contributing to the reduction of blood sugar level by harmoniously mixing only the active ingredients that are effective in suppressing blood sugar and reducing weight.

Next, in the third process S103, as shown in FIGS. 2A and 2B, the loess jar containing therein the blood sugar level-lowering mixture coated with the mineral mixture solution is heated to 30 to 50° C. for 12 to 24 hours to obtain an aged product.

As shown in FIG. 3, the loess jar containing therein the blood sugar level-lowering mixture coated with the mineral mixture solution is heated to 30 to 50° C. for 12 to 24 hours such that the blood sugar level-lowering mixture including the green tea is aged, thereby allowing the maximum extraction of the effective components of the materials in the activated state.

In this regard, the loess jar contains various mineral components, and thus far infrared rays, which are called the most effective rays of life for activating substances by heating, are emitted to affect the mixture of mineral minerals, and thus the action of ions becomes more active, thereby maximizing the aging of materials such as green tea and extracting the effects of the components to the maximum.

In particular, in the case of aging the materials in the loess jar, the long-term aging at room temperature or low temperature is general. However, in an embodiment of the present disclosure, the component efficacy may be maximized while maximizing the aging of the mixture in a short period of time such as 12 to 24 hours by heating the jar at 30 to 50° C.

Next, in the fourth process S104, the water is input into the jar at the same weight amount as a weight amount of the aged product to obtain an aged product mixed solution and then, the aged product mixed solution is pressurized to extract an extract concentrate.

Next, the fifth process S105 of diluting the extract concentrate with water at a ratio of 1:100 is executed to produce a diluted tea extract.

Thus, the user may drink the diluted tea extract at any time and before going to bed.

### Example

5 g of tourmaline generating ions in a powder form of 2 to 3 μm, 5 g of zeolite exchanging ions in a powder form of 2 to 3 μm, 5 g of sericite increasing the generation of ions in a powder form of 2 to 3 μm, and 1 g of illite maintaining ions balance in a powder form of 2 to 3 μm were mixed with each other in 984 g of water to obtain the mineral mixture solution. Then, the mineral mixture solution is maintained at room temperature for 30 hours. Then, the mineral mixture solution is diluted with water at a ratio of 10 g of the mineral mixture solution per 1 kg of the water to obtain a diluted mineral mixture solution.

Then, 3 kg of green tea, 2 kg of Jerusalem Artichoke, 1.5 kg of Lagerstroemia speciosa, 1.5 kg of Polygonatum odoratum, and 1 kg of Momordica charantia as main materials, and 200 g of of Schisandra chinensis, 200 g of Ophiopogon japonicus, 200 g of Nelumbo nucifera, 200 g of Cornus officinalis, 100 g of Wolfiporia extensa and 100 g of Asparagus cochinchinensis as auxiliary materials were mixed with each other to obtain the blood sugar level-lowering mixture. Then, 10 kg of the blood sugar level-lowering mixture was immersed in 100 kg of the diluted mineral mixture solution for 25 minutes, and is removed from the diluted mineral mixture solution to obtain the blood sugar level-lowering mixture coated with the mineral mixture solution. Then, 10 kg of the blood sugar level-lowering mixture coated with the mineral mixture solution is input into the loess jar.

Subsequently, the loess jar containing therein 10 kg of the blood sugar level-lowering mixture coated with the mineral mixture solution was heated to 45° C. for 20 hours to obtain the aged product.

Then, the water was input into the jar at the same weight amount as a weight amount of the aged product to obtain an aged product mixed solution and then, the aged product mixed solution is pressurized to extract an extract concentrate.

Then, the extract concentrate was diluted with water at a ratio of 1:100. In this way, the tea capable of lowering a blood sugar level was prepared as the diluted extract concentrate.

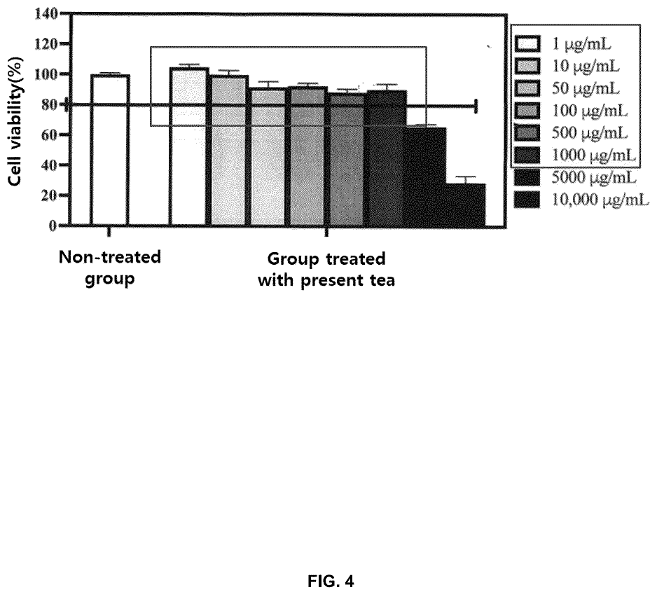

FIG. 4 is a diagram showing a relationship between cell viability based on a concentration of the tea capable of lowering a blood sugar level as prepared through the process of FIG. 1 in an experiment of transporting glucose into muscle cells of a mouse using the tea.

In the experiment of transporting glucose into muscle cells of mice, as shown in FIG. 4, the experimental concentration of the tea capable of lowering a blood sugar level as prepared through the process of FIG. 1 was set to 1 to 1000 μg/ml which exhibited the cell viability of 80% or greater, while the non-treated group exhibited 100% of the cell viability.

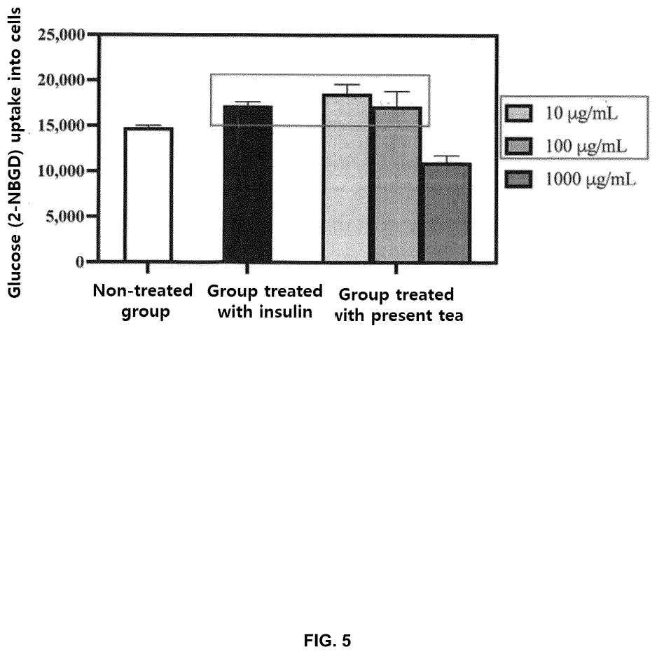

FIG. 5 is a diagram showing glucose absorption in the experiment of transporting glucose into the muscle cells of a mouse using insulin and the tea capable of lowering the blood sugar level as prepared through the process of FIG. 1.

In an experiment in which the effect of increasing glucose uptake in mouse muscle cells of the tea capable of lowering the blood sugar level as prepared through the process of FIG. 1 was compared with that using the insulin, a fluorescent substance was injected into glucose (2-NBDG), and the glucose (2-NBDG) was injected into the muscle cells of the mouse using the glucose transporter as each of the tea capable of lowering the blood glucose level prepared through the process of FIG. 1 and the insulin. Then, the fluorescence value in the muscle cells was measured. As shown in FIG. 5, the glucose (2-NBDG) uptake into the muscle cells was checked.

In this regard, it was identified that the amount by which the glucose (2-NBDG) was absorbed into the muscle cells is about 15,000 in the case of no treatment, and was about 17,000 in the case of insulin. Further, it was identified that the amount by which the glucose (2-NBDG) was absorbed into the muscle cells is about 18,000 in the case of 10 μ/ml of the concentration of the tea capable of lowering the blood sugar level prepared through the process of FIG. 1 and is 17,000 in the case of 100 μg/ml of the concentration of the tea capable of lowering the blood sugar level prepared through the process of FIG. 1, which are higher than or equal to the amount by which the glucose (2-NBDG) was absorbed into the muscle cells in the case of insulin.

Although this is the result used in muscle cell experiments, meaningful results can also be expected in experiments on the human body.

In general, when carbohydrate-based food enters the body, it is converted into glucose within 1 to 5 minutes, and in the pancreas, insulin, which acts as a key to the entry of glucose into cells, is produced, and glucose entered into cells is used as an energy source.

Such insulin is a proteinaceous hormone made in beta cells of Langerhans Island of the pancreas and secreted into the blood. The glucose absorption into the cells is not normally performed in a state in which the insulin is not active due to a lack of insulin amount or insulin activity in controlling the blood glucose level in the body. This problem can be solved by drinking the tea capable of lowering the blood glucose level as prepared through the process of FIG. 1 without inconvenience or side effects caused by the conventional use of insulin, zinc insulin, and blood glucose lowering agents.

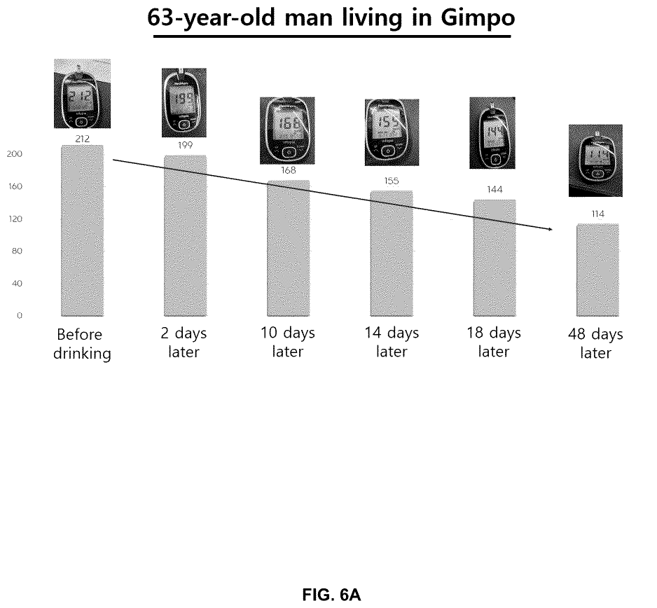

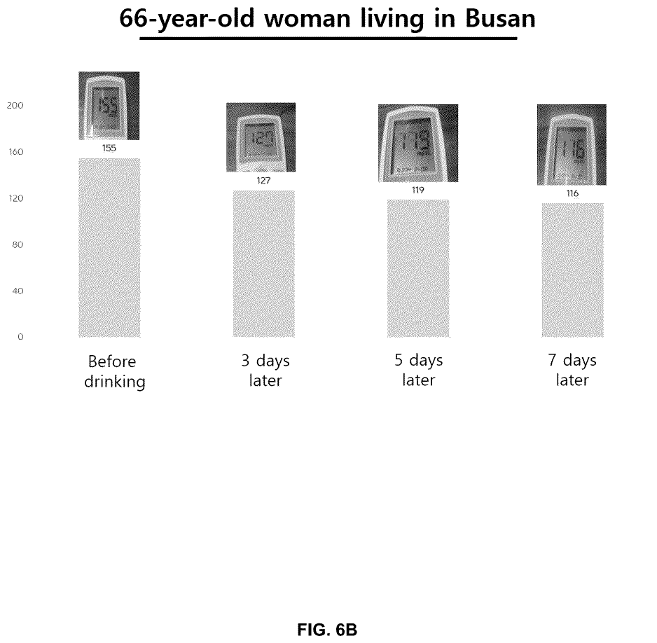

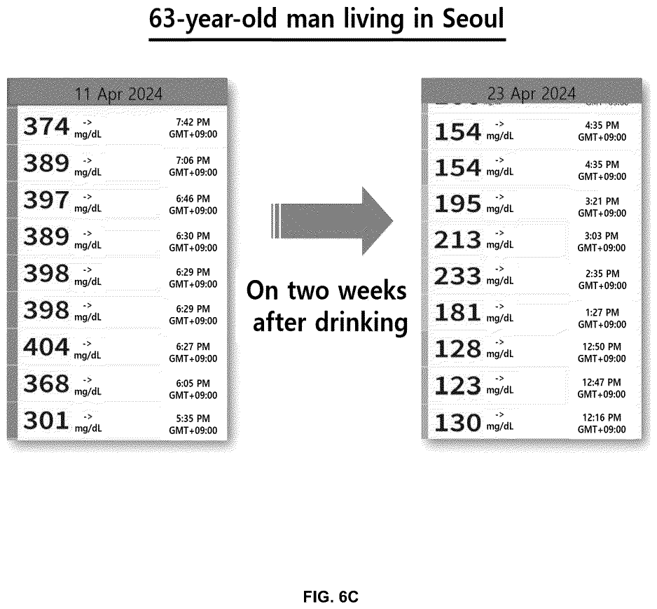

FIGS. 6A to 6C are diagrams illustrating an example of change in the blood glucose level before and after drinking the tea capable of lowering a blood sugar level as produced through the process of FIG. 1.

An experiment was conducted to check the effect of the tea capable of lowering the blood glucose level prepared by the method according to an embodiment of the present disclosure on diabetic patients. The tea capable of lowering the blood glucose level prepared through the process of FIG. 1 was frequently drunk and allowed to drink about 500 ml before going to bed.

First, as shown in FIG. 6A, a 65-year-old man living in Gimpo had a blood sugar level of 212 before the drinking, but on 2 days after the drinking, the blood sugar level was 199, on 10 days after the drinking, the blood sugar level was 166, on 14 days after the drinking, the blood sugar level was 155, on 18 days after the drinking, the blood sugar level was 144, and on 48 days after the drinking, the blood sugar level was lowered to 114.

Next, as shown in FIG. 6B, in the case of a 66-year-old woman residing in Busan, the blood sugar level before the drinking was 155, but the blood sugar level was lowered to 127 on 3 days after the drinking, 119 on 5 days after the drinking, and 116 on 7 days after the drinking.

Next, as shown in FIG. 6C, in the case of a 63-year-old man residing in Seoul, the blood glucose level before the drinking was 301 to 404, but on 2 weeks after the drinking, the blood glucose level was lowered to 123 to 195.

In the method for preparing a tea of capable of lowering a blood sugar level, the activation of the aging and the maximum extraction of effective ingredients of the mixture of materials that lower the blood sugar level may be achieved via ionic action of minerals and heating, thereby effectively lowering the blood sugar level of diabetic patients simply drinking the tea as the health functional food, and helping prevent diabetes and complications. Therefore, the method for preparing the tea of capable of lowering blood sugar level of the present disclosure can be used to prepare the tea as the functional food which can be well absorbed into the body of a diabetic patient, thereby relieving or eliminating the symptoms caused by insulin deficiency, and which can be conveniently drunk as the tea as the health functional food, thereby naturally reducing the blood sugar level, and thus preventing and alleviating diseases which may cause the abnormal blood sugar level.

Although the embodiments of the present disclosure have been described above with reference to the accompanying drawings, the present disclosure is not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above are not restrictive but illustrative in all respects.

What is claimed is:

1. A method for preparing a tea capable of lowering a blood sugar level, the method comprising:
a first process in which 0.1 to 1.0% by weight of tourmaline in a powder form of 2 to 3 μm, 0.1 to 1.0% by weight of zeolite in a powder form of 2 to 3 μm, 0.1 to 1.0% by weight of sericite in a powder form of 2 to 3 μm, and 0.05 to 0.5% by weight of illite in a powder form of 2 to 3 μm are mixed with each other in 96.5 to 99.65% by weight of water to obtain a mineral mixture solution, and then, the mineral mixture solution is maintained at room temperature for 24 to 36 hours, and then is diluted with water at a ratio of 10 to 15 g of the mineral mixture solution per 1 kg of the water to obtain a diluted mineral mixture solution;
a second process in which 30% by weight of green tea, 20% by weight of Jerusalem Artichoke, 15% by weight of Lagerstroemia speciosa, 15% by weight of Polygonatum odoratum, and 10% by weight of Momordica charantia as main materials, and 2% by weight of Schisandra chinensis, 2% by weight of Ophiopogon japonicus, 2% by weight of Nelumbo nucifera, 2% by weight of Cornus officinalis, 1% by weight of Wolfiporia extensa and 1% by weight of Asparagus cochinchinensis as auxiliary materials are mixed with each other to obtain a blood sugar level-lowering mixture, and then, the blood sugar level-lowering mixture is immersed in the diluted mineral mixture solution for 20 to 30 minutes, and is removed from the diluted mineral mixture solution to obtain the blood sugar level-lowering mixture coated with the mineral mixture solution, and then the blood sugar level-lowering mixture coated with the mineral mixture solution is input into a loess jar;
a third process of heating the loess jar containing therein the blood sugar level-lowering mixture coated with the mineral mixture solution to 30 to 50° C. for 12 to 24 hours to obtain an aged product; and
a fourth process in which water is input into the jar at the same weight amount as a weight amount of the aged product to obtain an aged product mixed solution and then, the aged product mixed solution is pressurized to extract an extract concentrate; and
a fifth process of diluting the extract concentrate with water at a ratio of 1:100.
